(19) United States
(12) Patent Application Publication
(10) Pub. No.: US 2022/0087870 A1
(43) Pub. Date: Mar. 24, 2022

(54) Absorbent Dressing With Indicator And Mechanical Decoupling Of Expansion Forces

(71) Applicant: KCI Licensing, Inc., San Antonio, TX (US)

(72) Inventors: Benjamin Andrew PRATT, Poole (GB); Matthew WESTMORELAND, North Yorkshire (GB); James Killingworth SEDDON, Wimborne (GB); Thomas Alan EDWARDS, Hampshire (GB); Christopher Brian LOCKE, Bournemouth (GB)

(21) Appl. No.: 17/422,250

(22) PCT Filed: Jan. 8, 2020
(86) PCT No.: PCT/US2020/012660

§ 371 Date: Jul. 12, 2021

Related U.S. Application Data
(60) Provisional application No. 62/798,159, filed on Jan. 29, 2019.

(51) Int. Cl. A61F 13/00; A61F 13/02

(52) U.S. Cl. CPC A61F 13/00068; A61F 13/0243; A61F 13/0226; A61F 13/0216

(57) ABSTRACT

A dressing for treating a tissue site, which may comprise a tissue interface and a cover comprising an expansion zone configured to be disposed over the tissue interface. The tissue interface may comprise a tissue contact layer having a treatment aperture in some examples. In more particular examples, the cover may be coupled to the tissue contact layer to form an expansion chamber between the expansion zone and the tissue contact layer. The cover may also comprise a base coupled to the tissue contact layer in some examples. Additionally, the tissue interface may further comprise an absorbent disposed within the expansion chamber in some embodiments. In some examples, the absorbent may be a manifold. Additionally or alternatively, the expansion chamber and the absorbent may be detachable.

### RELATED APPLICATIONS

The present application claims priority to U.S. Provisional Patent Application No. 62/798,159, entitled “Absorbent Dressing with Indicator and Mechanical Decoupling of Expansion Forces,” filed Jan. 29, 2019, which is incorporated herein by reference for all purposes.

### TECHNICAL FIELD

The invention set forth in the appended claims relates generally to tissue treatment systems and more particularly, but without limitation, to absorbent dressings with capacity indication and mechanical decoupling of expansion forces.

### BACKGROUND

Dressings are generally considered standard care for many types of tissue treatment, particularly for treating wounds. Regardless of the etiology of a wound, whether trauma, surgery, or another cause, proper care of the wound is important to the outcome. Dressings can provide many functions that can be beneficial for healing wounds, including controlling the wound environment and protecting a wound from bacteria and further physical trauma.

While the benefits of dressings for tissue treatment are widely known, improvements to dressings may benefit healthcare providers and patients.

### BRIEF SUMMARY

New and useful systems, apparatuses, and methods for treating a tissue site are set forth in the appended claims. Illustrative embodiments are also provided to enable a person skilled in the art to make and use the claimed subject matter.

For example, in some embodiments, a moist wound-healing foam dressing may provide a dressing-full indicator. The dressing may comprise a foam layer with a laminated backing film. The backing film may have a crease down the center of the dressing, and an indicator may be concealed under the crease. If the dressing reaches a predetermined saturation level of exudate, expansion of the foam can unfold the crease to reveal the indicator, which can indicate that the dressing should be changed to reduce the risk of maceration. In some examples, the indicator may also be a pH indicator, signifying bacterial colonization.

In other examples, an absorbent dressing may provide mechanical expansion, full-dressing indication, or both. Mechanical expansion may be provided by an expansion chamber created between a cover and a tissue contact layer. In some examples, the cover and the tissue contact layer may comprise a film, such as a polyurethane film, having a high moisture-vapor transfer rate. A perimeter of the cover and the tissue contact layer may be mechanically joined, such as with a weld or an adhesive. An absorbent may be placed within the chamber, and may be exposed through an aperture. The cover may provide relief geometry, such as corrugations, which can move outward and upward without transferring forces to the dressing perimeter. Change in the relief geometry can additionally provide an indication of dressing capacity.

More generally, some embodiments of a dressing for treating a tissue site may comprise a tissue interface and a cover comprising an expansion zone configured to be disposed over the tissue interface. The tissue interface may comprise a tissue contact layer having a treatment aperture in some examples. In more particular examples, the cover may be coupled to the tissue contact layer to form an expansion chamber between the expansion zone and the tissue contact layer. The cover may also comprise a base coupled to the tissue contact layer in some examples. Additionally, the tissue interface may further comprise an absorbent disposed within the expansion chamber in some embodiments. In some examples, the absorbent may be a manifold. Additionally or alternatively, the expansion chamber and the absorbent may be detachable.

In more particular examples, the tissue interface may additionally comprise a fluid control layer having a plurality of perforations, and the absorbent may be disposed adjacent to the plurality of perforations.

Additionally or alternatively, some embodiments of the tissue contact layer may comprise a bonding interface configured to adhere at least a portion of the tissue contact layer to epidermis adjacent to the tissue site. The tissue contact layer may comprise a sealing layer adjacent to the bonding interface, the sealing layer having a plurality of holes configured to expose portions of the bonding interface.

In other examples, a dressing for treating a tissue site may generally comprise an absorbent; a cover layer comprising an expansion zone over the absorbent; and an expansion indicator associated with the expansion zone. The expansion zone may be defined by a fold in the cover in some embodiments, and the expansion indicator may be disposed in the fold.

In some embodiments, a dressing for treating a tissue site may comprise a tissue contact layer; an expansion chamber adjacent to the tissue contact layer; and an absorbent disposed within expansion chamber and at least partially exposed through the tissue contact layer.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a schematic diagram of a dressing that can be used to treat tissue in accordance with this specification.

FIG. 2 is a schematic diagram illustrating the dressing of FIG. 1 in an expanded state.

FIG. 3 is a schematic diagram of another example of a dressing that can be used to treat a tissue site.

FIG. 4 is a schematic view of the dressing of FIG. 3 applied to a tissue site.

FIG. 5 is a top view of another example of a dressing.

FIG. 6 illustrates the dressing of FIG. 5 in an expanded state.

FIG. 7 is an assembly diagram of another example of a dressing.

FIG. 8 is an assembly diagram of another example of a dressing.

FIG. 9A illustrates an example application of the dressing of FIG. 7.

FIG. 9B illustrates removal of a portion of the dressing of FIG. 9A.

FIG. 10A illustrates another example application of the dressing of FIG. 7.

FIG. 10B illustrates removal of a portion of the dressing of FIG. 10A.

FIG. 11A illustrates an example application of the dressing of FIG. 8.

FIG. 11B illustrates removal of a portion of the dressing of FIG. 11A.

FIG. 12 is a schematic of an example embodiment of a therapy system that can provide negative-pressure therapy to a tissue site.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

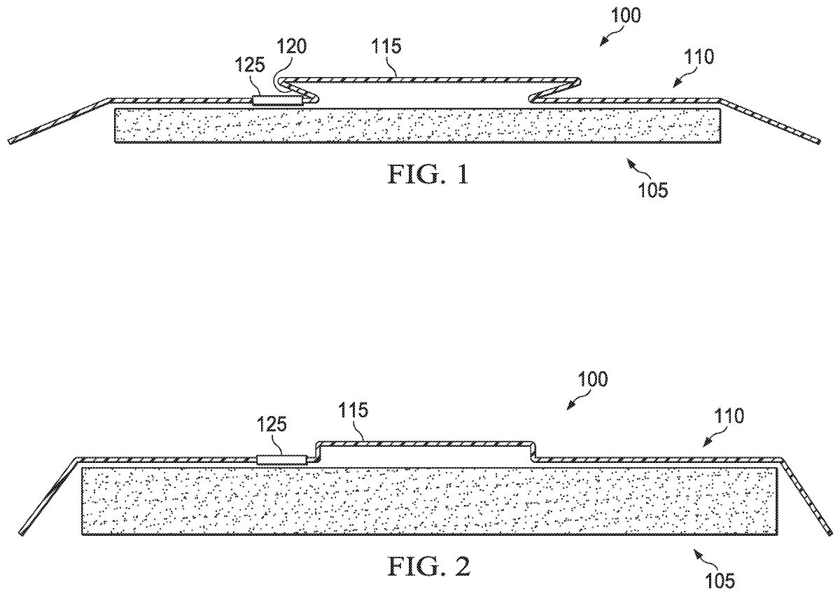

FIG. 1 is a schematic diagram of a dressing 100 that can be used to treat tissue in accordance with this specification. The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue.

As illustrated in the example of FIG. 1, the dressing 100 may comprise or consist essentially of a tissue interface 105 and a cover 110. The cover 110 may comprise an expansion zone 115 over the tissue interface 105. In the example of FIG. 1, the expansion zone 115 is defined by a fold 120 in the cover 110. An expansion indicator 125 may also be associated with some examples of the expansion zone 115. In FIG. 1, the expansion indicator 125 is disposed in the fold 120 on the cover 110, adjacent to the expansion zone 115. In some configurations, the expansion indicator 125 may be an adhesive label of contrasting color, text, pattern, or images, for example. In other examples, the contrasting color, text, pattern, or images may be printed directly on the cover 110.

The tissue interface 105 can be generally adapted to partially or fully contact a tissue site. The tissue interface 105 may take many forms, and may have many sizes, shapes, or thicknesses, depending on a variety of factors, such as the type of treatment being implemented or the nature and size of a tissue site. For example, the size and shape of the tissue interface 105 may be adapted to the contours of deep and irregular shaped tissue sites.

The thickness of the tissue interface 105 may also vary according to needs of a prescribed therapy. For example, the thickness of the tissue interface may be decreased to reduce tension on peripheral tissue. The thickness of the tissue interface 105 can also affect the conformability of the tissue interface 105. In some embodiments, a thickness in a range of about 5 millimeters to 10 millimeters may be suitable.

In some embodiments, the tissue interface 105 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 105 may further serve as a scaffold for new cell-growth, or a scaffold material may be used in conjunction with the tissue interface 105 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Suitable drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

Some embodiments of the dressing 100 may additionally include bonding interface, which may be used to attach the cover 110 to an attachment surface, such as undamaged epidermis, a gasket, or another cover. The bonding interface may take many forms. For example, a bonding interface may be a medically-acceptable, pressure-sensitive adhesive configured to bond the cover 110 to an attachment surface around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of a bonding interface may include a double-sided tape, paste, hydrocolloid, hydrogel, silicone gel, or organogel.

FIG. 2 is a schematic diagram illustrating the dressing 100 of FIG. 1 in an expanded state. In the example of FIG. 2, the tissue interface 105 may comprise or consist essentially of an absorbent that expands as it absorbs exudate or other liquid from a tissue site. The expansion zone 115 may be configured to allow the cover 110 to expand as the volume of the tissue interface 105 increases. Additionally, the expansion indicator 125 can be configured to indicate saturation of the tissue interface. For example, the expansion indicator 125 may be concealed by the fold 120 if the tissue interface 105 is not saturated, as illustrated in FIG. 1, and the expansion indicator 125 may be revealed as the tissue interface 105 expands the cover 110, as illustrated in FIG. 2.

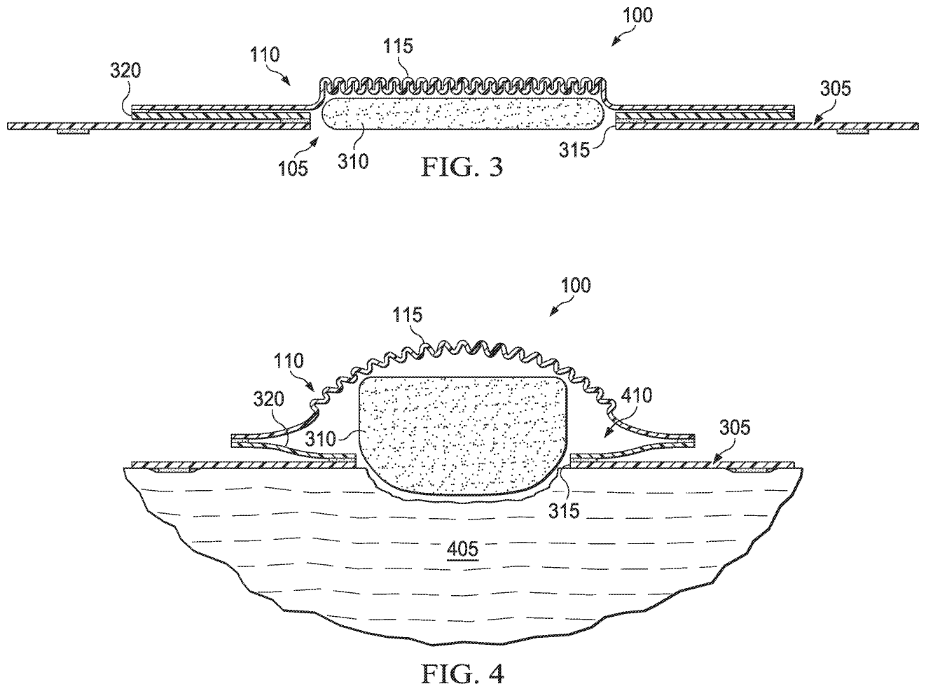

FIG. 3 is a schematic diagram of another example of the dressing 100, illustrating additional details that may be associated with some embodiments. As illustrated FIG. 3, some embodiments of the tissue interface 105 may comprises more than layer. In FIG. 3, the tissue interface 105 comprises a tissue contact layer 305 and an absorbent 310. The tissue interface 105 may also have a treatment aperture 315, as illustrated in FIG. 3, or may have a plurality of treatment apertures in some examples. In FIG. 3, the treatment aperture 315 is formed in the tissue contact layer 305. In some examples, the treatment aperture 315 may form a frame, window, or other opening around a surface of the absorbent 310.

The tissue contact layer 305 may be formed from a polymer film, such as a polyurethane film. In other examples, the tissue contact layer 305 may comprise or consist essentially of a hydrocolloid, hydrogel, or silicone gel. A pressure-sensitive adhesive or other bonding interface may be disposed on the tissue contact layer 305 in some examples.

In some examples, the absorbent 310 may be a super-absorbent polymer. The absorbent 310 may be disposed adjacent to the treatment aperture 315. For example, the absorbent 310 may be disposed in or over the treatment aperture 315.

As illustrated in FIG. 3, some embodiments of the expansion zone 115 may comprise or consist essentially of a corrugated portion of the cover 110, which can be disposed over or otherwise adjacent to the absorbent 310.

The cover 110 may be coupled to the tissue contact layer 305 in some embodiments. For example, as illustrated in FIG. 3, the cover 110 may comprise a base 320, which can be coupled to the tissue contact layer 305 in some embodiments. In some examples, the base 320 may be a ring adhered or welded to the tissue contact layer 305 around the treatment aperture 315. In some embodiments, the base 320 may be constructed from a material similar to the cover 110, such as a polyurethane film.

The dressing 100 may also have a release liner (not shown) in some embodiments, which may be configured to protect the tissue contact layer 305 and any adhesive prior to use. The release liner may be embossed in some examples. The release liner may comprise or consist essentially of a casting paper or a polymer film, for example. In some embodiments, the release liner may comprise or consist of a polyethylene film. Further, in some embodiments, the release liner may be a polyester material such as polyethylene terephthalate (PET), or similar polar semi-crystalline polymer. For example, a polar semi-crystalline polymer may be highly orientated and resistant to softening, swelling, or other deformation that may occur when brought into contact with components of the dressing 100, or when subjected to temperature or environmental variations, or sterilization. Further, a release agent may be disposed on a side of the release liner that is configured to contact the tissue contact layer 305. For example, the release agent may be a silicone coating and may have a release factor suitable to facilitate removal of the release liner by hand and without damaging or deforming the dressing 100. In some embodiments, the release agent may be a fluorocarbon or a fluorosilicone, for example. In other embodiments, the release liner may be uncoated or otherwise used without a release agent.

FIG. 4 is a schematic view of the dressing of FIG. 3 applied to a tissue site 405, illustrating additional details that may be associated with some examples. For example, FIG. 4 illustrates an expansion chamber 410 adjacent to the tissue contact layer 305. As illustrated in the example of FIG. 4, the expansion chamber 410 may be formed by the cover 110 coupled to the base 320. The absorbent 310 may be disposed within the expansion chamber 410, and may be at least partially exposed to the tissue site 405 through the tissue contact layer 305. For example, the absorbent 310 may be exposed through the treatment aperture 315 in some embodiments. FIG. 4 also illustrates the absorbent 310 in an expanded state, which may be caused by absorption of exudate or other liquid, for example. As the absorbent 310 expands into the expansion chamber 410, the absorbent 310 can expand the expansion zone 115, which can lift the cover 110. A peripheral portion of the base 320 can lift and separate from the tissue contact layer 305 without transferring any significant force to the tissue contact layer 305. Accordingly, the tissue contact layer 305 can maintain substantially the same contact area with an attachment surface adjacent to the tissue site 405. Additionally, the expansion zone 115 may comprise corrugations, which are spread as the expansion zone 115 expands and results in a visible change to the cover 110. For example, vertical surfaces of the expansion zone 115 that may be concealed in a dry state may become oblique or horizontal surfaces visible in an expanded state. The visible change can indicate a saturation level of the absorbent 310 in some examples. Color, text, images, texture, or other suitable features may additionally be used to enhance the visibility of the changes, or may be configured to indicate only a state of complete saturation.

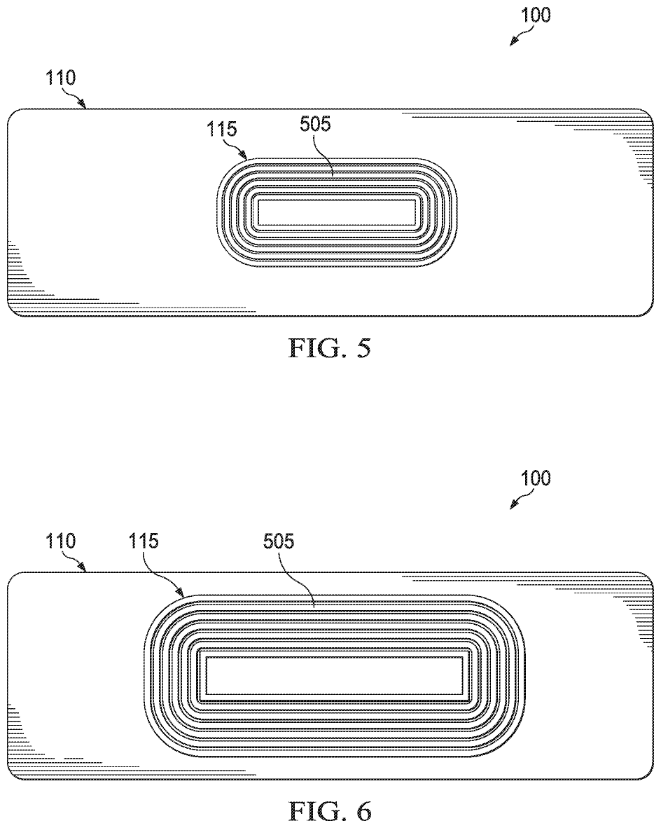

FIG. 5 is a top view of another example of the dressing 100, analogous to the dressing 100 of FIG. 4. In the example of FIG. 5, the cover 110 comprises corrugations 505 arranged in a concentric pattern that form the expansion zone 115.

FIG. 6 illustrates the dressing 100 of FIG. 5 in an expanded state, which may be caused by absorption of exudate or other liquid, for example. The corrugations 505 may spread as the expansion zone 115 expands, and can provide a visible change to the cover 110. The visible change can indicate a saturation level of an absorbent, such as the absorbent 310, beneath the cover 110. Color, text, images, texture, or other suitable features may additionally be used to enhance the visibility of the changes, or may be configured to indicate only a state of complete saturation. Additionally or alternatively, the corrugations 505 may expand to an extreme that presents the expansion zone 115 as a smooth surface, which can indicate saturation of the absorbent 310.

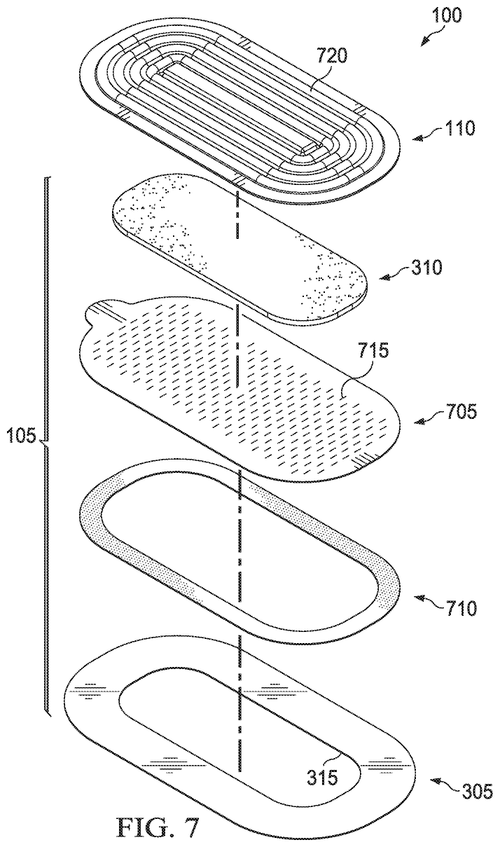

FIG. 7 is an assembly diagram of another example of the dressing 100, illustrating details that may be associated with some embodiments. In the example of FIG. 7, the tissue interface 105 comprises a fluid control layer 705, in addition to the tissue contact layer 305 and the absorbent 310. An adhesive gasket 710 may be disposed between the tissue contact layer 305 and the fluid control layer 705. When assembled, the adhesive gasket 710 can couple a periphery of the fluid control layer 705 to the tissue contact layer 305.

In FIG. 7, the fluid control layer 705 may comprise or consist essentially of a liquid-impermeable, elastomeric material. For example, the fluid control layer 705 may comprise or consist essentially of a polymer film, such as a polyurethane film. In some embodiments, the fluid control layer 705 may comprise or consist essentially of the same material as the cover 110. The fluid control layer 705 may also have a smooth or matte surface texture in some embodiments. A glossy or shiny finish better or equal to a grade B3 according to the SPI (Society of the Plastics Industry) standards may be particularly advantageous for some applications. In some embodiments, variations in surface height may be limited to acceptable tolerances. For example, the surface of the fluid control layer 705 may have a substantially flat surface, with height variations limited to 0.2 millimeters over a centimeter.

In some embodiments, the fluid control layer 705 may be hydrophobic. The hydrophobicity of the fluid control layer 705 may vary, but may have a contact angle with water of at least ninety degrees in some embodiments. For example, in some embodiments, the contact angle of the fluid control layer 705 may be in a range of at least 90 degrees to about 120 degrees, or in a range of at least 120 degrees to 150 degrees. The hydrophobicity of the fluid control layer 705 may be further enhanced with a hydrophobic coating of other materials, such as silicones and fluorocarbons, either as coated from a liquid, or plasma coated.

The area density of the fluid control layer 705 may vary according to a prescribed therapy or application. In some embodiments, an area density of less than 40 grams per square meter may be suitable, and an area density of about 20-30 grams per square meter may be particularly advantageous for some applications.

In some embodiments, the fluid control layer 705 may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

As illustrated in the example of FIG. 7, the fluid control layer 705 may have one or more fluid restrictions 715, which can be distributed uniformly or randomly across the fluid control layer 705. The fluid restrictions 715 may be bi-directional and pressure-responsive. For example, each of the fluid restrictions 715 generally may comprise or consist essentially of an elastic passage that is normally unstrained to substantially reduce liquid flow, and can expand or open in response to a pressure gradient. In some embodiments, the fluid restrictions 715 may comprise or consist essentially of perforations or fenestrations in the fluid control layer 705. Perforations may be formed by cutting through the fluid control layer 705, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid restrictions 715 may be an elastomeric valve that is normally closed when unstrained to substantially prevent liquid flow, and can open in response to a pressure gradient.

For example, some embodiments of the fluid restrictions 715 may comprise or consist essentially of one or more slits, slots or combinations of slits and slots in the fluid control layer 705. In some examples, the fluid restrictions 715 may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

In FIG. 7, the cover 110 comprises a plurality of ridges 720 arranged in a concentric pattern that form the expansion zone 115. The ridges 720 may expand as the expansion zone 115 expands, which can provide a visible change to the cover 110. The visible change can indicate a saturation level of an absorbent, such as the absorbent 310, beneath the cover 110. Color, text, images, texture, or other suitable features may additionally be used to enhance the visibility of the changes, or may be configured to indicate only a state of complete saturation. Additionally or alternatively, the ridges 720 may expand to an extreme that presents the expansion zone 115 as a smooth surface, which can indicate saturation of the absorbent 310.

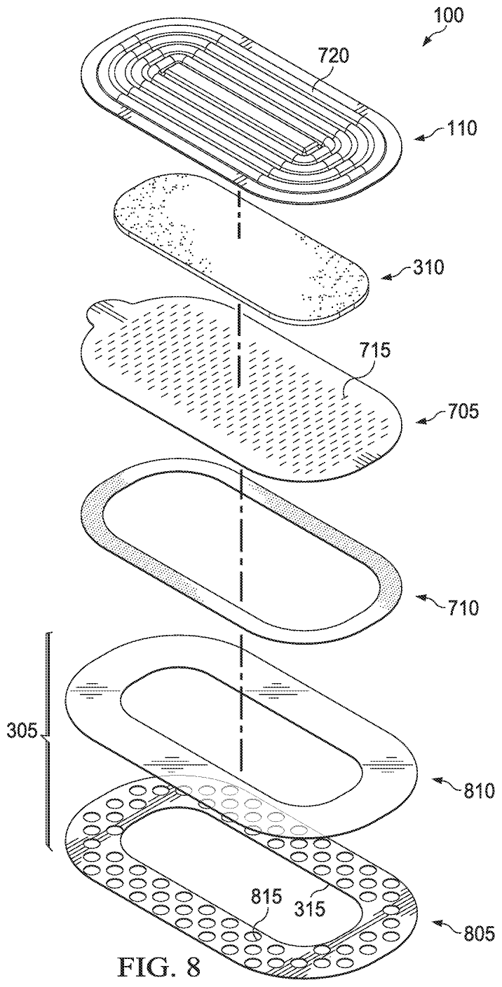

FIG. 8 is an assembly diagram of another example of the dressing 100, illustrating details that may be associated with some embodiments. In the example of FIG. 8, the tissue contact layer 305 comprises a sealing layer 805 and a bonding interface 810.

In some examples, the sealing layer 805 may be formed from a soft, pliable material suitable for providing a fluid seal with a tissue site, such as a suitable gel material, and may have a substantially flat surface. For example, the sealing layer 805 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. In some embodiments, the sealing layer 805 may have a thickness between about 200 microns (μm) and about 1000 microns (μm). In some embodiments, the sealing layer 805 may have a hardness between about 5 Shore OO and about 80 Shore OO. Further, the sealing layer 805 may be comprised of hydrophobic or hydrophilic materials.

In some embodiments, the sealing layer 805 may be a hydrophobic-coated material. For example, the sealing layer 805 may be formed by coating a porous material, such as, for example, woven, nonwoven, molded, or extruded mesh with a hydrophobic material. The hydrophobic material for the coating may be a soft silicone, for example.

The sealing layer 805 may have apertures 815 disposed around the treatment aperture 315. In some examples, as illustrated in FIG. 8, the treatment aperture 315 may be symmetrical and centrally disposed in the sealing layer 805, forming an open central window.

The apertures 815 may be formed by cutting, perforating, or by application of local RF or ultrasonic energy, for example, or by other suitable techniques for forming an opening or perforation in the sealing layer 805. The apertures 815 may have a uniform distribution pattern, or may be randomly distributed in the sealing layer 805. The apertures 815 may have many shapes, including circles, squares, stars, ovals, polygons, slits, complex curves, rectilinear shapes, triangles, for example, or may have some combination of such shapes.

Each of the apertures 815 may have uniform or similar geometric properties. For example, in some embodiments, each of the apertures 815 may be circular apertures, having substantially the same diameter. In some embodiments, each of the apertures 815 may have a diameter of about 1 millimeter to about 50 millimeters. In other embodiments, the diameter of each of the apertures 815 may be about 1 millimeter to about 20 millimeters. In some embodiments, geometric properties of the apertures 815 may vary. For example, the diameter of the apertures 815 may vary depending on the position of the apertures 815 in the sealing layer 805.

At least one of the apertures 815 may be positioned at the edges of the sealing layer 805, and may have an interior cut open or exposed at the edges that is in fluid communication in a lateral direction with the edges. The lateral direction may refer to a direction toward the edges and in the same plane as the sealing layer 805.

The bonding interface 810 may be disposed between the sealing layer 805 and the adhesive gasket 710 in some examples. The bonding interface 810 may comprise a carrier, which may be formed from the same or similar material as the cover 110 in some embodiments. For example, a carrier may comprise or consist essentially of a polymer film, such as a polyurethane film. The bonding interface 810 may additionally include an adhesive, which may be disposed on the carrier. The adhesive may be used to attach the bonding interface 810 to an attachment surface, such as undamaged epidermis, a gasket, or another cover, through one or more of the apertures 815 in the sealing layer 805. In some examples, the adhesive may be a medically-acceptable, pressure-sensitive adhesive configured to bond to an attachment surface around a tissue site. An acrylic adhesive may be suitable for some embodiments, and the adhesive may have a coating weight of about 25-65 grams per square meter (g.s.m.) in some examples. Thicker adhesives, or combinations of adhesives, may be applied in some embodiments to improve the seal and reduce leaks. Other example embodiments of the bonding interface 810 may include a double-sided tape, paste, hydrocolloid, hydrogel, silicone gel, or organogel.

In some embodiments, the bonding interface 810 may be substantially coextensive with the sealing layer 805. When assembled, the bonding interface 810 may adhere to the sealing layer 805, and the adhesive gasket 710 can couple a periphery of the fluid control layer 705 to the bonding interface 810.

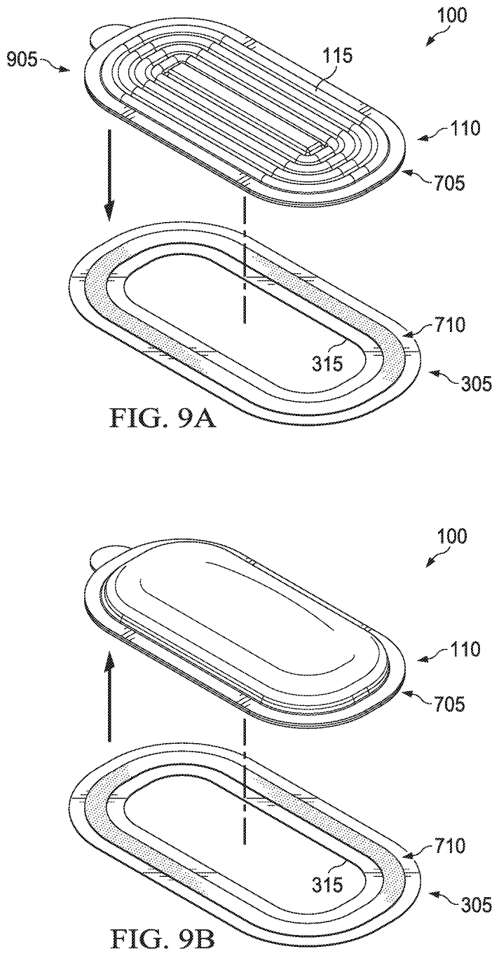

FIG. 9A illustrates an example application of a fluid management assembly 905 to the tissue contact layer 305 of FIG. 7. In the example of FIG. 9A, the fluid management assembly 905 is assembled from the cover 110, the absorbent 310 (not visible), and the fluid control layer 705 of FIG. 7, and the adhesive gasket 710 is coupled to the tissue contact layer 305. The expansion zone 115 in FIG. 9A is relaxed, indicating the fluid management assembly 905 is new, unused, dry, or otherwise unsaturated. FIG. 9B illustrates removal of the fluid management assembly 905 from the adhesive gasket 710 and the tissue contact layer 305 of FIG. 9A. For example, the fluid management assembly 905 may be removed when the expansion zone 115 is expanded as in FIG. 9B, indicating the fluid management assembly 905 is saturated. In FIG. 9B, the adhesive gasket 710 remains attached to the tissue contact layer 305 if the fluid management assembly 905 is removed. For example, the adhesive gasket 710 may have a bond strength with the tissue contact layer 305 that is greater than the bond strength with the fluid management assembly 905.

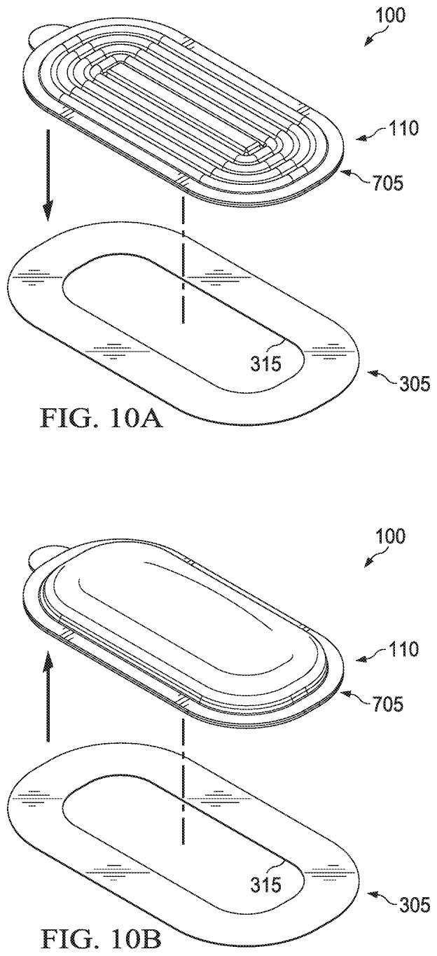

FIG. 10A illustrates another application of the fluid management assembly 905, in which the adhesive gasket 710 (not visible) is coupled to the fluid management assembly 905. The fluid management assembly 905 (and the adhesive gasket 710) may be coupled to the tissue contact layer 305 as illustrated in FIG. 10A, and may be removed as illustrated in FIG. 10B. In the example of FIG. 10B, the adhesive gasket 710 may have a bond strength with the fluid management assembly 905 that is greater than the bond strength with the tissue contact layer 305, so that the adhesive gasket 710 remains attached to the fluid management assembly 905 if the fluid management assembly 905 is removed.

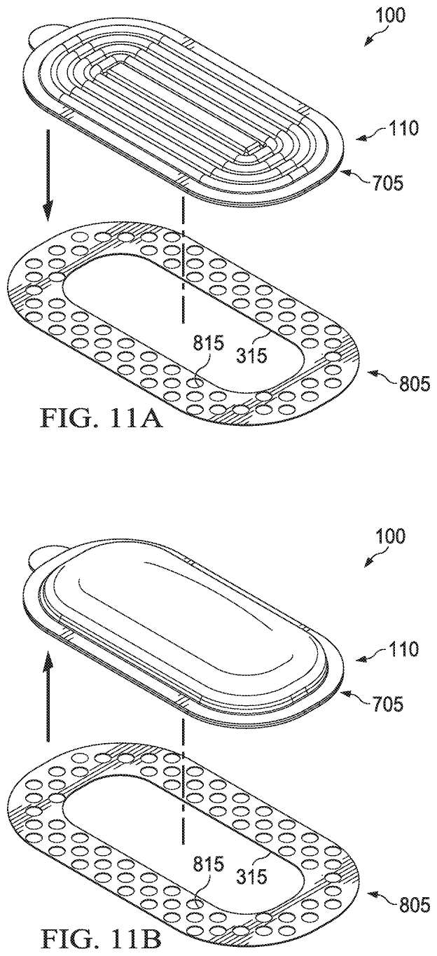

FIG. 11A and FIG. 11B illustrate another example configuration in which the fluid management assembly 905 is applied to the tissue contact layer 305 of FIG. 8. The adhesive gasket 710 may be coupled to either the fluid management assembly 905 or the tissue contact layer 305.

Advantageously, in examples such as FIGS. 9A-9B, FIGS. 10A-10B, and FIGS. 11A-11B, the fluid management assembly 905 may be removed and replaced without removing the tissue contact layer 305, which can significantly reduce or eliminate trauma associated with dressing changes and reduce costs.

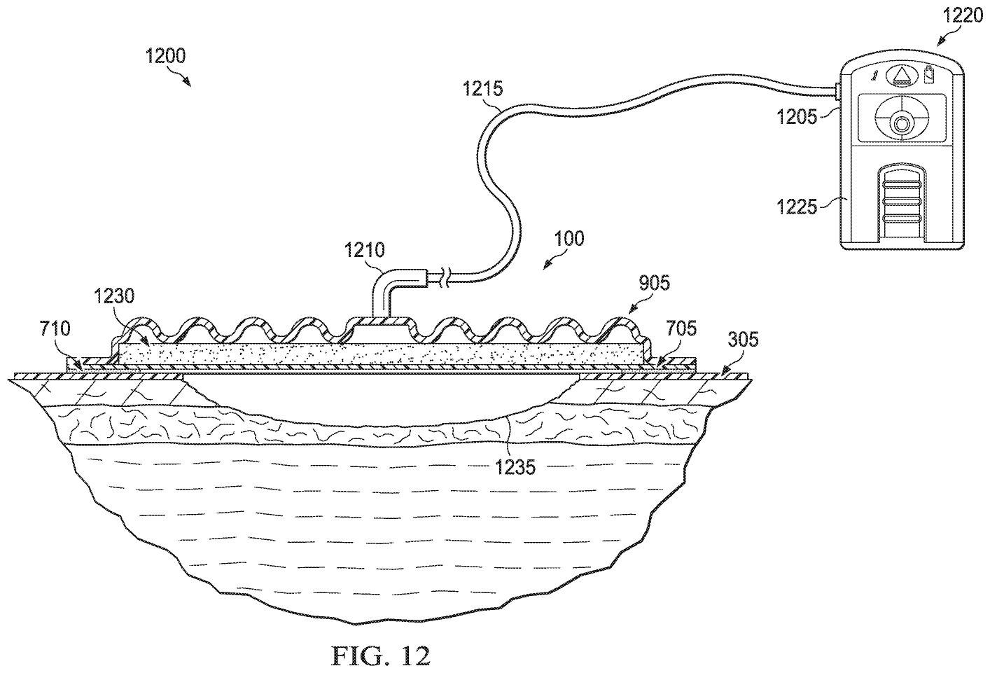

FIG. 12 is a schematic of an example embodiment of a therapy system 1200 that can provide negative-pressure therapy to a tissue site with various embodiments of the dressing 100.

The therapy system 1200 may include a source or supply of negative pressure, such as a negative-pressure source 1205, and one or more distribution components. A distribution component is preferably detachable and may be disposable, reusable, or recyclable. The dressing 100 is an example of a distribution component that may be associated with the therapy system 1200.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface 1210 may facilitate coupling a fluid conductor 1215 to the dressing 100. For example, the dressing interface 1210 may be a SENSAT.R.A.C.™ Pad available from Kinetic Concepts, Inc. of San Antonio, Tex.

The therapy system 1200 may also include a regulator or controller in some examples. Additionally, the therapy system 1200 may include sensors to measure operating parameters and provide feedback signals to the controller indicative of the operating parameters.

Some components of the therapy system 1200 may be housed within or used in conjunction with other components, such as sensors, processing units, alarm indicators, memory, databases, software, display devices, or user interfaces that further facilitate therapy. For example, in some embodiments, the negative-pressure source 1205 may be combined with a controller and other components into a therapy unit 1220.

In general, components of the therapy system 1200 may be coupled directly or indirectly. Coupling may include fluid, mechanical, thermal, electrical, or chemical coupling (such as a chemical bond), or some combination of coupling in some contexts. For example, the negative-pressure source 1205 may be electrically coupled to a controller and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 1205, may be an electrically-powered a vacuum pump. In other examples, a suitable negative-pressure source may be a manual pump, a reservoir of air at a negative pressure, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 1205 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The therapy system 1200 may also include a container, canister, pouch, or other storage component, which can be used to manage exudates and other fluids withdrawn from a tissue site. As illustrated in the example of FIG. 12, a container 1225 may be incorporated into the therapy unit 1220. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A suitable controller may be a microprocessor or computer programmed to operate one or more components of the therapy system 1200, such as the negative-pressure source 1205. In some embodiments, for example, the controller may be a microcontroller, which generally comprises an integrated circuit containing a processor core and a memory programmed to directly or indirectly control one or more operating parameters of the therapy system 1200. Operating parameters may include the power applied to the negative-pressure source 1205, the pressure generated by the negative-pressure source 1205, or the pressure distributed to the dressing 100, for example. The controller may also be configured to receive one or more input signals, such as a feedback signal, and programmed to modify one or more operating parameters based on the input signals.

Sensors are generally known in the art as any apparatus operable to detect or measure a physical phenomenon or property, and generally provide a signal indicative of the phenomenon or property that is detected or measured. For example, sensors may be configured to measure one or more operating parameters of the therapy system 1200. In some embodiments, the therapy system 1200 may have one or more sensors that are a transducer configured to measure pressure in a pneumatic pathway and convert the measurement to a signal indicative of the pressure measured. In some embodiments, for example, one or more of the sensors may be a piezo-resistive strain gauge. Additionally or alternatively, one or more sensors may optionally measure operating parameters of the negative-pressure source 1205, such as a voltage or current, in some embodiments. Preferably, the signals from the sensors are suitable as an input signal to a controller, but some signal conditioning may be appropriate in some embodiments. For example, the signal may need to be filtered or amplified before it can be processed by a controller. Typically, the signal is an electrical signal, but may be represented in other forms, such as an optical signal.

In some embodiments, the tissue interface 105 may comprise a manifold, instead of or in addition to the absorbent 310. In some embodiments, the absorbent 310 may be a manifold. In the example of FIG. 12, the tissue interface 105 comprises a manifold 1230, in addition to tissue contact layer 305 and the fluid control layer 705. A manifold in this context may comprise or consist essentially of a means for collecting or distributing fluid across a tissue interface under pressure. For example, a manifold may be adapted to receive negative pressure from a source and distribute negative pressure through multiple apertures across a tissue interface, which may have the effect of collecting fluid from across a tissue site and drawing the fluid toward the source. In some embodiments, the fluid path may be reversed or a secondary fluid path may be provided to facilitate delivering fluid, such as instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the manifold 1230 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the manifold 1230 may also vary according to needs of a prescribed therapy. The 25% compression load deflection of the manifold 1230 may be at least 0.35 pounds per square inch, and the 65% compression load deflection may be at least 0.43 pounds per square inch. In some embodiments, the tensile strength of the manifold 1230 may be at least 10 pounds per square inch. The manifold 1230 may have a tear strength of at least 2.5 pounds per inch. In some embodiments, the manifold 1230 may be foam comprised of polyols such as polyester or polyether, isocyanate such as toluene diisocyanate, and polymerization modifiers such as amines and tin compounds. In some examples, the manifold 1230 may be reticulated polyurethane foam such as found in GRANUFOAM™ dressing or V.A.C. VERAFLO™ dressing, both available from Kinetic Concepts, Inc. of San Antonio, Tex.

The manifold 1230 may be either hydrophobic or hydrophilic. In an example in which the manifold 1230 may be hydrophilic, the manifold 1230 may also wick fluid away from a tissue site, while continuing to distribute negative pressure to the tissue site. The wicking properties of the manifold 1230 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In operation, the dressing 100 may be placed within, over, on, or otherwise proximate to a tissue site 1235. If the tissue site 1235 site is a wound, for example, the dressing 100 may be placed over the wound. In some examples, the cover 110 may be placed over the manifold 1230 and the fluid control layer 705 and sealed to an attachment surface near a tissue site. For example, the cover 110 may be sealed to the adhesive gasket 710. In other examples, the fluid management assembly 905 may be applied to the adhesive gasket 710 or to the tissue contact layer 305. Thus, the dressing 100 can provide a sealed therapeutic environment proximate to the tissue site 1235, substantially isolated from the external environment, and the negative-pressure source 1205 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site 1235 through the dressing 100 can induce macro-strain and micro-strain in the tissue site in the sealed therapeutic environment. Negative pressure can also remove exudate and other fluid from a tissue site, which can be collected in the container 1225.

In some embodiments, a controller associated with the therapy unit 1220 may control the operation of one or more components of the therapy system 1200 to manage the pressure delivered to the dressing 100. In some embodiments, a controller may include an input for receiving a desired target pressure and may be programmed for processing data relating to the setting and inputting of the target pressure to be applied to the dressing 100. In some example embodiments, the target pressure may be a fixed pressure value set by an operator as the target negative pressure desired for therapy at a tissue site and then provided as input to the controller. The target pressure may vary from tissue site to tissue site based on the type of tissue forming a tissue site, the type of injury or wound (if any), the medical condition of the patient, and the preference of the attending physician. After selecting a desired target pressure, a controller can operate the negative-pressure source 1205 in one or more control modes based on the target pressure and may receive feedback from one or more sensors to maintain the target pressure at the dressing 100.

In some embodiments, a controller may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by a controller, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

The systems, apparatuses, and methods described herein may provide significant advantages. For example, some examples of the dressing 100 can reduce the risk of maceration to a tissue site with a clear indicator. The indicator can provide an objective indication of dressing capacity, substantially reducing or eliminating personal judgment and variability in knowing if a dressing should be changed before dressing failure. Additionally or alternatively, some examples of the dressing 100 can expand to accommodate rapid increase in size that may occur as fluid is stored in the dressing, while ensuring the dressing remains in place and reducing or eliminating discomfort to patients.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 100, the container 1225, or both may be separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site, the dressing comprising:
a tissue interface; and
a cover comprising an expansion zone configured to be disposed over the tissue interface.

2. The dressing of claim 1, wherein:
the tissue interface comprises a tissue contact layer having a treatment aperture;
the cover is coupled to the tissue contact layer to form an expansion chamber between the expansion zone and the tissue contact layer.

3. (canceled)

4. The dressing of claim 1, wherein the expansion zone comprises concentric pleats or corrugations in the cover.

5. (canceled)

6. The dressing of claim 2, wherein the cover further comprises a base coupled to the tissue contact layer.

7. The dressing of claim 2, wherein:
the tissue interface further comprises an absorbent disposed within the expansion chamber; and
the absorbent is at least partially exposed through the treatment aperture.

8. The dressing of claim 2, wherein:
the tissue interface further comprises a fluid control layer having a plurality of perforations; and
an absorbent disposed adjacent to the plurality of perforations.

9. The dressing of claim 2, wherein the tissue contact layer comprises a bonding interface configured to adhere at least a portion of the tissue contact layer to epidermis adjacent to the tissue site.

10. The dressing of claim 9, wherein the tissue contact layer further comprises a sealing layer adjacent to the bonding interface, the sealing layer having a plurality of holes configured to expose portions of the bonding interface.

11. The dressing of claim 9, wherein the bonding interface comprises:
a carrier; and
an adhesive disposed on the carrier.

12. The dressing of claim 11, wherein the carrier and the cover comprise a polymer film.

13. The dressing of claim 11, wherein the carrier comprises a treatment aperture.

14. The dressing of claim 10, wherein the sealing layer comprises a silicone gel.

15. The dressing of claim 9, wherein the cover and an absorbent are detachable from the bonding interface.

16. The dressing of claim 7, wherein the absorbent comprises one or more of a manifold, an open-cell foam, or a super-absorbent polymer.

17. (canceled)

18. (canceled)

19. The dressing of claim 1, further comprising an expansion indicator associated with the expansion zone.

20. A dressing for treating a tissue site, the dressing comprising:
an absorbent;
a cover layer comprising an expansion zone over the absorbent; and
an expansion indicator associated with the expansion zone.

21. The dressing of claim 20, wherein:
the expansion zone is defined by a fold in the cover; and
the expansion indicator is disposed in the fold.

22. The dressing of claim 20, wherein:
the expansion zone is defined by a fold in the cover; and
the expansion indicator comprises a color indicator disposed in the fold.

23. A dressing for treating a tissue site, the dressing comprising:
a tissue contact layer;
an expansion chamber adjacent to the tissue contact layer; and
an absorbent disposed within expansion chamber and at least partially exposed through the tissue contact layer.

24. An apparatus for treating a tissue site with negative pressure, the apparatus comprising:
a dressing according to claim 1; and
a negative-pressure source configured to deliver negative pressure to the tissue interface.

25. (canceled)
